(19) United States
(12) Patent Application Publication
(10) Pub. No.: US 2026/0083415 A1
(43) Pub. Date: Mar. 26, 2026

(54) RADIATION DETECTOR, IMAGING SYSTEM, AND TEMPERATURE CONTROL METHOD

(71) Applicant: GE Precision Healthcare LLC, Waukesha, WI (US)

(72) Inventor: Xinfeng Li, Beijing (CN)

(21) Appl. No.: 19/340,580

(22) Filed: Sep. 25, 2025

(30) Foreign Application Priority Data
Sep. 24, 2024 (CN) 202411356344.6

(51) Int. Cl. A61B 6/00; G01K 3/00; G01T 1/20

(52) U.S. Cl. CPC A61B 6/4488; G01K 3/005; G01T 1/20188

(57) ABSTRACT

The present disclosure relates to a radiation detector, an imaging system, and a temperature control method. The radiation detector includes a detector module. The detector module is used to detect X-rays. The radiation detector further includes a support frame, used to mount the detector module. The support frame performs heat conduction with the detector module. The radiation detector further includes a temperature regulation module. The temperature regulation module performs heat conduction with the support frame, and the temperature regulation module is used to perform heat conduction with a mounting plate on which the detector module is mounted. The temperature regulation module includes a temperature regulation element that uses electricity for cooling and heating.

### TECHNICAL FIELD

This application claims priority to Chinese Application No. 202411356344.6, filed on Sep. 26, 2024, the disclosure of which is incorporated herein by reference in its entirety.

### TECHNICAL FIELD

The present disclosure relates to the field of detection, and in particular, to a radiation detector, an imaging system including the radiation detector, and a temperature control method for the radiation detector.

### BACKGROUND

A detection system (for example, an imaging system) may be configured to image an examination subject and obtain a corresponding detection result. For example, computed tomography (CT) systems are widely used in various medical institutions to perform three-dimensional imaging on a region of interest, such as a lung, of the examination subject, so as to aid clinicians in accurate medical diagnosis of the examination subject.

Some detection systems use X-rays emitted by a radiation source to irradiate the examination subject, and use a detector on the side opposite to the radiation source to perform detection, so as to further analyze data and obtain information of the examination subject. For example, CT systems use X-rays emitted by an X-ray source to scan an examination subject (for example, a human body), receive the X-rays transmitted through the human body by using the detector, convert the X-rays into digital signals, and then form images by means of computer processing and analysis to obtain, for example, images of one or a plurality of parts of the human body.

A radiation detector is an important component of an imaging system (for example, a CT imaging system). The radiation detector should have heat stability, such that the quality and accuracy of an output signal (for example, an image) can meet the desired requirements.

### SUMMARY

In order to solve at least the described technical problems and/or other technical problems set forth in the present disclosure and/or other possible technical problems not set forth in the present disclosure, some embodiments of the present disclosure provide a radiation detector. The radiation detector includes a detector module, the detector module being used to detect rays; a support frame, used to mount the detector module, the support frame performing heat conduction with the detector module; and a temperature regulation module, the temperature regulation module performing heat conduction with the support frame, the temperature regulation module being used to perform heat conduction with a mounting plate on which the detector module is mounted, and the temperature regulation module including a temperature regulation element that uses electricity for cooling and heating.

In some examples, the temperature regulation element includes a thermoelectric cooler (TEC). In some examples, the temperature regulation module is disposed between the detector module and the mounting plate, the temperature regulation module is connected to the support frame and conducts heat, the temperature regulation module includes a metal heat-conducting structure on which the temperature regulation element is mounted, the metal heat-conducting structure performs heat conduction with the mounting plate, and the metal heat-conducting structure is thermally isolated from the support frame. In some examples, the metal heat-conducting structure is fixedly connected to the support frame, the temperature regulation element is provided on a portion of a surface of the metal heat-conducting structure facing the support frame, and a heat-insulating material is provided on at least part of a remaining portion of the surface of the metal heat-conducting structure, so as to thermally isolate the metal heat-conducting structure from the support frame. In some examples, the temperature regulation element is welded to the portion of the surface of the metal heat-conducting structure facing the support frame.

In some examples, the radiation detector further includes a temperature sensor, the temperature sensor being configured to measure the temperature of the detector module. The temperature regulation module is configured to: perform heating when the temperature of the detector module is lower than a preset temperature, so as to increase the temperature of the detector module to the preset temperature; and perform cooling when the temperature of the detector module is higher than the preset temperature, so as to reduce the temperature of the detector module to the preset temperature. In some examples, the preset temperature is greater than or equal to a maximum temperature allowed inside an imaging system employing the radiation detector. In some examples, the temperature regulation module is configured to allow a predetermined switching time to pass when switching between heating and cooling. In some examples, the radiation detector further includes at least one of: a first elastic heat-conducting pad disposed between the mounting plate and the temperature regulation module; and a second elastic heat-conducting pad disposed between the support frame and the temperature regulation module.

In some examples, the temperature regulation module is fixedly connected to the support frame at a first side, and the temperature regulation module is directly connected to the mounting plate at a second side. In some examples, the temperature regulation module is fixedly connected to the support frame at a first side, the support frame is connected to the mounting plate by means of a guide rail, and a surface of a second side of the temperature regulation module is in contact with the mounting plate, wherein the support frame is fixedly connected to the guide rail by means of a first fastener, and the guide rail is fixedly connected to the mounting plate by means of a second fastener. In some examples, a heat-insulating material is included between the support frame and the guide rail, and a heat-insulating material is included between the guide rail and the mounting plate, wherein the thickness of the heat-insulating material between the guide rail and the mounting plate is set such that a surface of the first side of the temperature regulation module is in contact with the mounting plate. In some examples, the detector module includes a flat plate-shaped detector circuit board, a detector element mounted on a surface of a side of the detector circuit board facing a ray source generating rays, and a signal processing element mounted on a surface of a side of the detector circuit board facing away from the ray source; the detector circuit board is mounted on the support frame, and the support frame includes a flat plate-shaped main body portion covering the detector module and performing heat conduction with the detector module.

In some examples, at least a portion of the outer perimeter of at least one of the temperature regulation module and the support frame is covered with a heat-insulating material. In some examples, the radiation detector further includes a heat-conducting pipe connected to the support frame. One end of the heat-conducting pipe is adjacent to the temperature regulation element, and the heat-conducting pipe includes a liquid and is configured to conduct heat generated by the temperature regulation element to the support frame.

The present disclosure further provides an imaging system, including a radiation source, the radiation source being configured to emit radiation rays; any radiation detector described above; and a gantry, wherein the radiation detector is disposed inside the gantry by means of the mounting plate.

The present disclosure further provides a temperature control method for any radiation detector described above, including starting, when the temperature of the detector module is lower than a preset temperature, the temperature regulation module to perform heating, so as to increase the temperature of the detector module to the preset temperature; and starting, when the temperature of the detector module is higher than the preset temperature, the temperature regulation module to perform cooling, so as to reduce the temperature of the detector module to the preset temperature. In some examples, the temperature control method further includes setting the preset temperature of the detector module to be greater than or equal to a maximum temperature allowed inside an imaging system employing the radiation detector. In some examples, the temperature control method further includes configuring the temperature regulation module to allow a predetermined switching time to pass when switching between heating and cooling.

### BRIEF DESCRIPTION OF THE DRAWINGS

In order to further describe embodiments of the present invention, the embodiments of the present invention will be presented in detail with reference to the accompanying drawings. It should be understood that these accompanying drawings may delineate only typical embodiments of the present invention, and thus will not be considered to limit the scope of protection claimed by the present invention.

In addition, the drawings show a main connection relationship or relative position relationship of each component, rather than all of these relationships, and the components and connections in the drawings are not necessarily drawn to scale in practice.

FIG. 1 shows an exemplary CT imaging system;

FIG. 2 is a block diagram of an exemplary imaging system similar to the CT imaging system in FIG. 1;

FIG. 3 is a schematic diagram of a radiation detector according to some embodiments of the present disclosure;

FIG. 4 is a schematic diagram of a temperature regulation module included in a radiation detector according to some embodiments of the present disclosure;

FIG. 5 is a schematic cross-sectional diagram of a radiation detector in a mounted state according to some embodiments of the present disclosure;

FIG. 6 is a three-dimensional schematic diagram of a radiation detector in a mounted state according to some embodiments of the present disclosure; and

FIG. 7 shows a temperature control method for a radiation detector according to some embodiments of the present disclosure.

### DETAILED DESCRIPTION

The following detailed description is provided with reference to the accompanying drawings. The accompanying drawings illustrate, via examples, specific embodiments capable of implementing the claimed subject matter. It should be understood that the following specific embodiments are intended to specifically describe typical examples for the purpose of explanation, but should not be understood as limiting the present invention. Given a full understanding of the spirit and gist of the present invention, a person skilled in the art can make appropriate modifications and adjustments to the disclosed embodiments without departing from the spirit and scope of the claimed subject matter of the present invention.

In the following detailed description, numerous specific details are set forth in order to provide a thorough understanding of the embodiments described. However, it will be apparent to those of ordinary skill in the art that the described various embodiments can be implemented without these specific details. In other examples, commonly-known structures are not described in detail to avoid unnecessarily obscuring aspects of the embodiments. Unless otherwise defined, terms used herein shall have the same meanings as commonly understood by those of ordinary skill in the art to which the present disclosure belongs.

The terms “first”, “second”, and the like, in the description and claims of the present disclosure do not denote any order, quantity, or importance, but are merely intended to distinguish between different constituents or features.

Embodiments of the present disclosure are exemplary implementations or examples. Reference in the description to “embodiments”, “one embodiment”, “some embodiments”, “alternative embodiments”, or “other embodiments” means that specific features and structures described with reference to embodiments are included in at least some embodiments of the present technology, but are not necessarily included in all embodiments. Various occurrences of “embodiments”, “one embodiment”, or “some embodiments” do not necessarily refer to the same embodiment. Elements or aspects from one embodiment may be combined with elements or aspects of another embodiment.

The term “and/or” in descriptions of the present disclosure describes only an association relationship between associated objects and represents that three relationships may exist. For example, A and/or B may indicate three situations, i.e., A exists alone, A and B exist simultaneously, or B exists alone. In addition, the character “/” in this specification generally indicates an “or”relationship between the associated objects.

Unless defined otherwise, technical terms or scientific terms used in the claims and description should have the usual meanings that are understood by those of ordinary skill in the technical field to which the present invention belongs. The terms “include” or “comprise” and similar words indicate that an element or object preceding the terms “include” or “comprise” encompasses elements or objects and equivalent elements thereof listed after the terms “include” or “comprise”, and do not exclude other elements or objects.

It should be understood that the description of the positions and directions in the present description is provided with reference to specific embodiments shown in the accompanying drawings, and is therefore a relative position description. In other embodiments where the placement direction of the device and apparatus is opposite or different than the direction shown in the drawings, these position descriptions may vary accordingly.

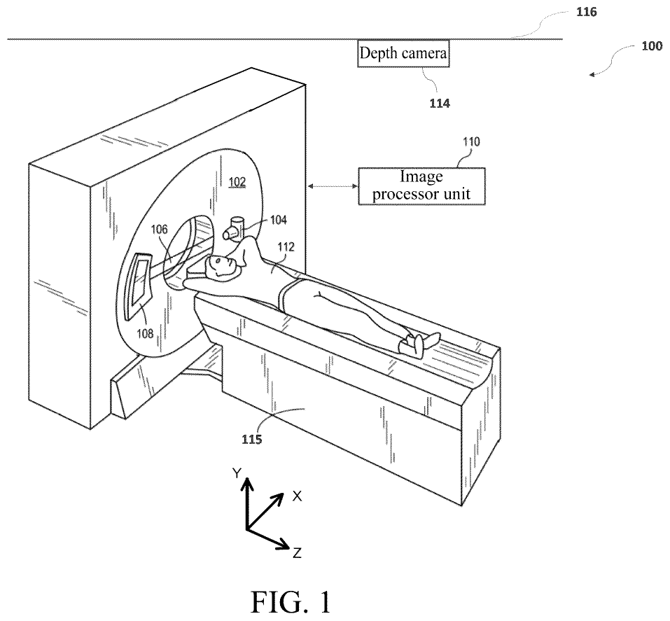

An exemplary radiation detector, an imaging system including the radiation detector, and a temperature control method for the radiation detector, which can be used to put the present invention into practice, will be described below with reference to FIG. 1 to FIG. 7.

Although in the present disclosure, the technology of the present invention is described in combination with a CT imaging apparatus, it should be understood that the technology of the present invention may also be applied to any other suitable type of imaging system, including, but not limited to, a baggage X-ray machine, various medical imaging systems, and the like. In addition to CT, the medical imaging systems may include other medical imaging modalities, such as a C-arm imaging system, a positron emission tomography (PET) system, a single photon emission computed tomography (SPECT) system, an interventional imaging system (such as angiography and biopsy), an X-ray radiation imaging system, an X-ray fluoroscopy imaging system, etc., and a combination thereof (for example, a multi-modality imaging system, such as a PET/CT or SPECT/CT imaging system). Different types of imaging systems are applicable for detection of corresponding objects. The object may be any type of suitable object. As an example, a baggage x-ray machine is suitable for detecting specific articles in baggage. For the medical imaging system, detectable objects include interventional objects (such as needles, endoscopes, implants, catheters, guide wires, dilators, ablators, contrast agents, etc.), lesions (such as tumors, etc.), bones, organ tissue structures, vascular structures, etc. In another aspect, for example, in addition to being used in the medical field, the CT imaging system may be used for, for example, part inspection and the like in the manufacturing industry.

FIG. 1 shows an exemplary CT imaging system 100. Specifically, the CT imaging system (also referred to as a CT apparatus) 100 is configured to image an examination subject 112 (such as a patient, an inanimate subject, one or a plurality of manufactured components, an industrial component, a foreign subject, or the like). Throughout the present disclosure, the terms “examination subject” and “patient” may be used interchangeably, and it should be understood that, at least in some embodiments, a patient is a type of examination subject that may be imaged by the CT imaging system 100, and that an examination subject may include a patient. In some embodiments, the CT imaging system 100 includes a gantry 102. The gantry 102 may include at least one X-ray radiation source 104. The at least one X-ray radiation source 104 is configured to project an X-ray beam (or X-ray) 106 for imaging the examination subject 112. Specifically, the X-ray radiation source 104 is configured to project the X-ray 106 toward a detector array 108 positioned on the opposite side of the gantry 102. Although FIG. 1 illustrates only one X-ray radiation source 104, in some embodiments, a plurality of X-ray radiation sources 104 may be used to project a plurality of X-rays 106 toward a plurality of detectors, so as to acquire projection data corresponding to the examination subject 112 at different energy levels.

In some embodiments, the X-ray radiation source 104 projects the fan-shaped or cone-shaped X-ray beam 106. The fan-shaped or cone-shaped X-ray beam 106 is collimated to be located in an x-y plane of a Cartesian coordinate system, and the plane is generally referred to as an “imaging plane” or a “scanning plane”. The X-ray beam 106 passes through the examination subject 112. The X-ray beam 106, after being attenuated by the examination subject 112, is incident on the detector array 108. The intensity of the attenuated radiation beam received at the detector array 108 depends on the attenuation of the X-ray 106 by the examination subject 112. Each detector element of the detector array 108 produces a separate electrical signal that serves as a measure of the intensity of the beam at the detector position.

Intensity measurements from all detectors are separately acquired to generate a transmission distribution.

In third-generation CT imaging systems, the gantry 102 is used to rotate the X-ray radiation source 104 and the detector array 108 within the imaging plane around the examination subject 112, so that the angle at which the X-ray beam 106 intersects with the examination subject 112 is constantly changing. A full gantry rotation occurs when the gantry 102 completes a full 360-degree rotation. A set of X-ray attenuation measurements (e.g., projection data) from the detector array 108 at one gantry angle is referred to as a “view”. Thus, the view represents each incremental position of the gantry 102. A “scan” of the examination subject 112 includes a set of views made at different gantry angles or viewing angles during one rotation of the X-ray radiation source 104 and the detector array 108.

In an axial scan, projection data is processed to construct an image corresponding to a two-dimensional slice captured through the examination subject 112. A method for reconstructing an image from a set of projection data is referred to as a filtered back projection technique in the art. The method converts an attenuation measurement from a scan into an integer referred to as “CT number” or “Hounsfield unit” (HU), the integer being used to control, for example, the brightness of a corresponding pixel on a cathode ray tube display.

In some examples, the CT imaging system 100 may include a depth camera 114 positioned on or outside the gantry 102. As shown in FIG. 1, the depth camera 114 is mounted on a ceiling panel 116 positioned above the examination subject 112 and oriented to image the examination subject when the examination subject 112 is at least partially outside the gantry 102. The depth camera 114 may include one or more light sensors, including one or more visible light sensors and/or one or more infrared (IR) light sensors. In some implementations, the one or more IR sensors may include one or more sensors in a near-IR range and a far-IR range to implement thermal imaging. In some embodiments, the depth camera 114 may further include an IR light source. The light sensor may be any 3D depth sensor, such as a time-of-flight (ToF) sensor, a stereo sensor, or a structured light depth sensor, the 3D depth sensor being operable to generate a 3D depth image, while in other embodiments the light sensor may be a two-dimensional (2D) sensor operable to generate a 2D image. In some such implementations, a 2D light sensor may be used to infer a depth from knowledge of light reflection to estimate a 3D depth. Regardless of whether the light sensor is a 3D depth sensor or a 2D sensor, the depth camera 114 may be configured to output a signal for encoding an image to a suitable interface.

The interface may be configured to receive, from the depth camera 114, the signal for encoding the image. In other examples, the depth camera 114 may further include other components, such as a microphone, so that the depth camera can receive and analyze directional and/or non-directional sound from the observed examination subject and/or other sources.

In some embodiments, the CT imaging system 100 further includes an image processing unit 110 configured to reconstruct an image of a target volume of a patient by using a suitable reconstruction method (such as an iterative or analytical image reconstruction method). For example, the image processing unit 110 may reconstruct an image of a target volume of a patient by using an analytical image reconstruction method (such as filtered back projection (FBP)). As another example, the image processing unit 110 may reconstruct an image of a target volume of a patient by using an iterative image reconstruction method (such as adaptive statistical iterative reconstruction (ASIR), conjugate gradient (CG), maximum likelihood expectation maximization (MLEM), model-based iterative reconstruction (MBIR), or the like).

As used herein, the phrase “reconstructing an image” is not intended to exclude an embodiment of the present invention in which data representing an image is generated rather than a viewable image. Thus, as used herein, the term “image” broadly refers to both a viewable image and data representing a viewable image. However, many embodiments generate (or are configured to generate) at least one viewable image.

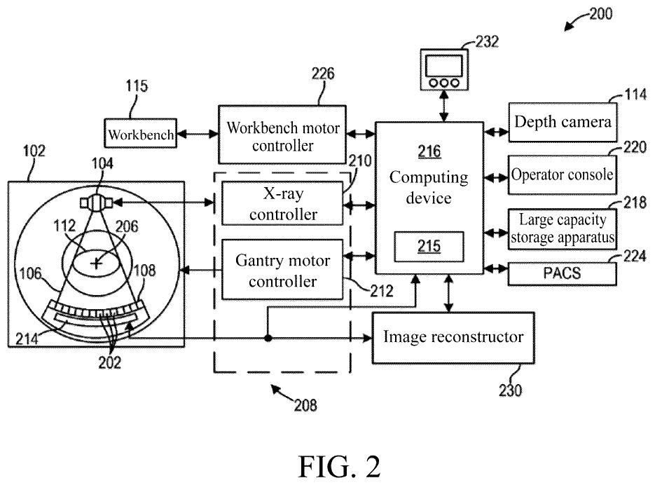

The CT imaging system 100 further includes a workbench 115, and the examination subject 112 is positioned on the workbench 115 to facilitate imaging. The workbench 115 may be electrically powered, so that a vertical position and/or a lateral position of the workbench can be adjusted. Accordingly, the workbench 115 may include a motor and a motor controller, as will be explained below with respect to FIG. 2. The workbench motor controller moves the workbench 115 by adjusting the motor, so as to properly position the examination subject in the gantry 102 to acquire projection data corresponding to a target volume of the examination subject. The workbench motor controller may adjust the height of the workbench 115 (e.g., a vertical position relative to a floor on which the workbench is located) and a lateral position of the workbench 115 (e.g., a horizontal position of the workbench along an axis parallel to an axis of rotation of the gantry 102).

FIG. 2 shows an exemplary imaging system 200 similar to the CT imaging system 100 in FIG. 1. In some embodiments, the imaging system 200 includes the detector array 108 (see FIG. 1). The detector array 108 further includes a plurality of detector elements 202. The plurality of detector elements together collect the X-ray beam 106 (see FIG. 1) passing through the examination subject 112 to acquire corresponding projection data. Therefore, in some embodiments, the detector array 108 is fabricated in a multi-slice configuration including a plurality of rows of units or detector elements 202. In such configurations, one or more additional rows of detector elements 202 are arranged in a parallel configuration for acquiring projection data. In some examples, an individual detector in the detector array 108 or the detector elements 202 may include a photon counting detector that registers interactions of individual photons into one or more energy bins. It should be understood that the methods described herein may also be implemented using an energy integration detector.

In some embodiments, the imaging system 200 is configured to traverse different angular positions around the examination subject 112 to acquire required projection measurement data. Therefore, the gantry 102 and components mounted thereon can be configured to rotate about a center of rotation 206 to acquire, for example, projection measurement data at different energy levels. Alternatively, in embodiments in which a projection angle with respect to the examination subject 112 changes over time, the mounted components may be configured to move along a substantially curved line rather than a segment of a circumference.

In some embodiments, the imaging system 200 includes a control mechanism 208 to control the movement of the components, such as the rotation of the gantry 102 and the operation of the X-ray radiation source 104. In some embodiments, the control mechanism 208 further includes an X-ray controller 210, configured to provide power and timing signals to the X-ray radiation source 104. Additionally, the control mechanism 208 includes a gantry motor controller 212, configured to control the rotational speed and/or position of the gantry 102 on the basis of imaging requirements.

In some embodiments, the control mechanism 208 further includes a data acquisition system (DAS) 214, configured to sample analog data received from the detector elements 202, and convert the analog data to a digital signal for subsequent processing. The data sampled and digitized by the DAS 214 is transmitted to a computer or computing device 216. In an example, the computing device 216 stores data in a storage apparatus 218. For example, the storage apparatus 218 may include a hard disk drive, a floppy disk drive, a compact disc-read/write (CD-R/W) drive, a digital versatile disc (DVD) drive, a flash drive, and/or a solid-state storage drive.

Additionally, the computing device 216 provides commands and parameters to one or more of the DAS 214, the X-ray controller 210, and the gantry motor controller 212 to control system operations, such as data acquisition and/or processing. In some implementations, the computing device 216 controls system operations on the basis of operator input. The computing device 216 receives the operator input by means of an operator console 220 that is operably coupled to the computing device 216, the operator input including, for example, commands and/or scan parameters. The operator console 220 may include a keyboard (not shown) or a touch screen to allow the operator to specify commands and/or scan parameters.

Although FIG. 2 shows only one operator console 220, more than one operator console may be coupled to the imaging system 200, for example, for inputting or outputting system parameters, requesting examination, and/or viewing images. Moreover, in some embodiments, the imaging system 200 may be coupled to, for example, a plurality of displays, printers, workstations, and/or similar devices located locally or remotely within an institution or hospital or in a completely different location by means of one or more configurable wired and/or wireless networks (such as the Internet and/or a virtual private network).

In some embodiments, for example, the imaging system 200 includes or is coupled to a picture archiving and communication system (PACS) 224. In one exemplary embodiment, the PACS 224 is further coupled to a remote system (such as a radiology information system or a hospital information system), and/or an internal or external network (not shown) to allow operators in different locations to provide commands and parameters and/or acquire access to image data.

The computing device 216 uses operator-provided and/or system-defined commands and parameters to operate a workbench motor controller 226, which can in turn control a workbench motor, thereby adjusting the position of the workbench 115 shown in FIG. 1. Specifically, the workbench motor controller 226 moves the workbench 115 by means of the workbench motor, so as to properly position the examination subject 112 in the gantry 102 to acquire projection data corresponding to a target volume of the examination subject 112. For example, the computing device 216 may send a command to the workbench motor controller 226 to instruct the workbench motor controller 226 to adjust the vertical position and/or the lateral position of the workbench 115 by means of the motor.

As described previously, the DAS 214 samples and digitizes the projection data acquired by the detector elements 202. Subsequently, an image reconstructor 230 uses the sampled and digitized X-ray data to perform high-speed reconstruction. Although the image reconstructor 230 is shown as a separate entity in FIG. 2, in some embodiments, the image reconstructor 230 may form a part of the computing device 216. Alternatively, the image reconstructor 230 may not be present in the imaging system 200, and the computing device 216 may instead perform one or more functions of the image reconstructor 230. In addition, the image reconstructor 230 may be located locally or remotely and may be operably connected to the imaging system 200 by using a wired or wireless network. Specifically, in one exemplary embodiment, computing resources in a “cloud” network cluster are available to the image reconstructor 230.

In some embodiments, the image reconstructor 230 stores the reconstructed image in the storage apparatus 218. Alternatively, the image reconstructor 230 transmits the reconstructed image to the computing device 216 to generate usable examination subject information (also referred to as examination subject information) for diagnosis and evaluation. In some embodiments, the computing device 216 transmits the reconstructed images and/or examination subject information to a display 232, and the display is communicatively coupled to the computing device 216 and/or the image reconstructor 230. In some embodiments, the display 232 allows an operator to evaluate an imaged anatomical structure. The display 232 may further allow the operator to select a volume of interest (VOI) and/or request examination subject information by means of, for example, a graphical user interface (GUI) for subsequent scanning or processing.

As described further herein, the computing device 216 may include computer-readable instructions, and the computer-readable instructions are executable to send, according to an examination imaging scheme, commands and/or control parameters to one or more of the DAS 214, the X-ray controller 210, the gantry motor controller 212, and the workbench motor controller 226. The examination imaging scheme includes a clinical task/intent, also referred to herein as a clinical intent identifier (CID) of the examination. For example, the CID may inform of a goal (e.g., a general scan or lesion detection, an anatomical structure of interest, a quality parameter, or another goal) of the procedure on the basis of a clinical indication, and may further define the position and orientation (e.g., posture) of the examination subject required during a scan (e.g., supine and feet first). The operator of the system 200 may then position the examination subject on the workbench according to the examination subject position and orientation specified by the imaging scheme. Further, the computing device 216 may set and/or adjust various scan parameters (e.g., a dose, a gantry rotation angle, kV, mA, an attenuation filter) according to the imaging scheme. For example, the imaging scheme may be selected by the operator from a plurality of imaging schemes stored in a memory on the computing device 216 and/or a remote computing device, or the imaging scheme may be automatically selected by the computing device 216 according to received examination subject information.

During the examination/scanning phase, it may be desirable to expose the examination subject to a radiation dose as low as possible while still maintaining the required the quality of images. In addition, reproducible and consistent imaging quality between examinations and between examination subjects, as well as between different imaging system operators, may be required. Thus, an imaging system operator may manually adjust the position of the workbench and/or the position of the examination subject, so as to, for example, center a desired anatomical structure of a patient at the center of a gantry bore. However, such a manual adjustment may be error-prone. Therefore, the CID associated with the selected imaging scheme may be mapped to various positioning parameters of the examination subject. The positioning parameters of the examination subject include the posture and orientation of the examination subject, the height of the workbench, an anatomical reference for scanning, and a starting and/or ending scan position.

Thus, the depth camera 114 may be operably and/or communicatively coupled to the computing device 216 to provide image data to determine the anatomy of the examination subject, including the posture and orientation. Additionally, various methods and procedures described further herein for determining the patient anatomy on the basis of image data generated by the depth camera 114 may be stored as executable instructions in a non-transitory memory of the computing device 216.

Additionally, in some examples, the computing device 216 may include a camera image data processor 215 that includes instructions for processing information received from the depth camera 114. The information (which may include depth information and/or visible light information) received from the depth camera 114 may be processed to determine various parameters of the examination subject, such as the identity of the examination subject, the physique (e.g., the height, weight, and patient thickness) of the examination subject, and the current position of the examination subject relative to the workbench and the depth camera 114. For example, prior to imaging, the body contour or anatomy of the examination subject 112 may be estimated by using images reconstructed from point cloud data, and the point cloud data is generated by the camera image data processor 215 according to images received from the depth camera 114. The computing device 216 may use these parameters of the examination subject to perform, for example, patient-scanner contact prediction, scan range superposition, and scan key point calibration, as will be described in further detail herein. Further, data from the depth camera 114 may be displayed by means of the display 232.

In some embodiments, information from the depth camera 114 may be used by the camera image data processor 215 to perform tracking of one or a plurality of examination subjects in the field of view of the depth camera 114. In some examples, skeleton tracking may be performed by using image information (e.g., depth information), in which a plurality of joints of the examination subject are identified and analyzed to determine the motion, posture, position, etc., of the examination subject. The positions of joints during the skeleton tracking can be used to determine the above-described parameters of the examination subject. In other examples, the image information may be directly used to determine the above-described parameters of the examination subject without skeleton tracking.

On the basis of these positioning parameters of the examination subject, the computing device 216 may output one or a plurality of alerts to the operator regarding patient posture/orientation and examination (e.g., scan) result prediction, thereby reducing the possibility that the examination subject is exposed to a higher than desired radiation dose and improving the quality and reproducibility of the image generated by the scan. As an example, the estimated body structure may be used to determine whether the examination subject is in an imaging position specified by the radiologist, thereby reducing the incidence of repeating the scan due to improper positioning. Furthermore, the amount of time an imaging system operator spends positioning the examination subject can be reduced, allowing more scans to be performed per day and/or allowing additional interaction with the examination subject.

A plurality of exemplary patient orientations may be determined on the basis of data received from a depth camera (such as the depth camera 114 described in FIG. 1 and FIG. 2). For example, a controller (e.g., the computing device 216 in FIG. 2) may perform patient structure extraction and posture estimation on the basis of an image received from the depth camera 114, thereby enabling different patient orientations to be distinguished from each other.

The CT imaging system 100 may perform imaging examination on the basis of a scanning protocol. The scanning protocol is a description of the imaging examination. The scanning protocol may include a description of an involved body part, for example, a medical or colloquial term for the body part. The scanning protocol may provide various parameters and related information for performing scans and post-processing, such as a power value, the duration of radiation, speed of movement, radiation energy, and a time delay between image captures, etc. It is conceivable that any configurable technical parameter that should be used for imaging examination by the imaging system 110 may be defined in the scanning protocol.

The CT imaging system 100 may have an automatic patient positioning function. That is, a patient may be automatically positioned in a scan start position in an opening of the gantry 102 on the basis of an examination instruction or the scanning protocol, and moved in the Z-axis direction to a scan end position during scanning and imaging. A conventional automatic patient positioning function may automatically determine the scan range in the horizontal direction on the basis of the anatomical structure to be imaged (e.g., from an examination instruction or the scanning protocol) and the patient structure from the depth camera 114, but the automatic centering thereof can only be substantially for the head or the body and the average body contour center of all scout scan ranges, so the precision of centering for particular anatomical structures and special patients is not good enough.

As mentioned above, temperature stability has an important impact on the imaging quality of an imaging system. At present, in design processes for radiation detectors of some imaging systems (for example, CT imaging systems), because the designed number of rows of radiation detectors is increasing, the radiation detectors require more power when working; accordingly, heat generated also increases, causing the temperature of the radiation detectors to increase, which poses a greater challenge to the temperature stability of the radiation detectors. When the power of a radiation detector increases, a large heat sink needs to be selected to dissipate heat from the radiation detector. Existing radiation detectors with a heat sink have a complex design structure. In addition, because the required heat sink is large, the power of a heater needs to be greatly increased if it is necessary to maintain the temperature stability of the radiation detector at, for example, a low temperature. This may cause problems in many aspects. In one aspect, a large amount of heat can be quickly transferred to the surrounding air with, for example, large-area heat dissipation fins of a large heat sink, which increases energy consumption of the system. In another aspect, as the power required by the radiation detector increases, accuracy of control of the temperature of the detector may be reduced.

Embodiments of the present disclosure provide a radiation detector, which can solve at least one of the above technical problems. The radiation detector according to some embodiments of the present disclosure can also solve further technical problems and achieve further technical effects, as described in detail below.

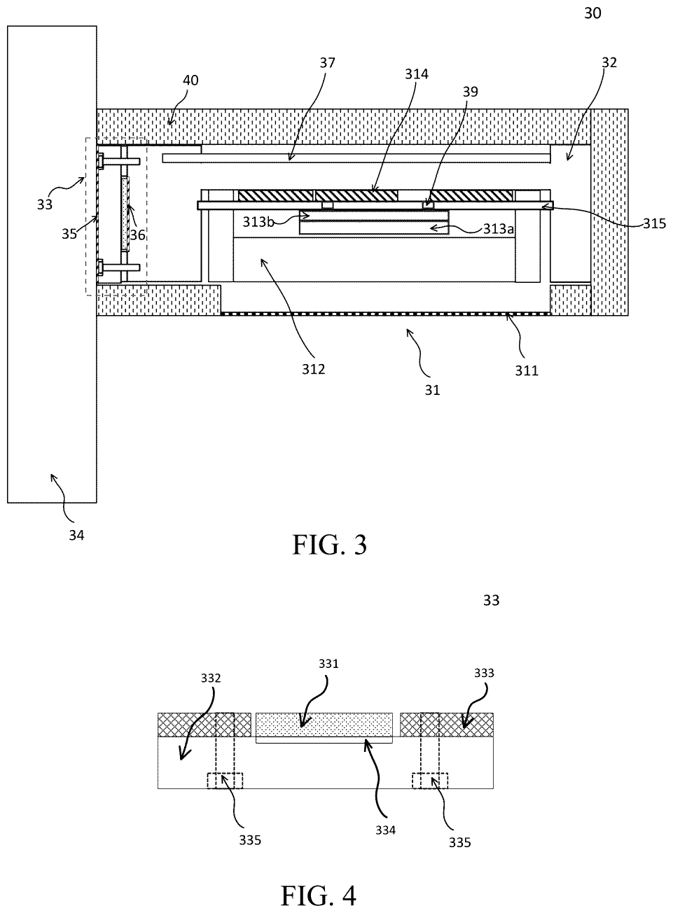

FIG. 3 is a schematic diagram of a radiation detector 30 according to some embodiments of the present disclosure. In some embodiments of the present disclosure, the radiation detector 30 may correspond to, for example, the detector element 202 described above with reference to FIG. 2, but the radiation detector 30 in FIG. 3 integrates one or more of functions such as signal detection, signal conversion, signal processing, and temperature control. For the sake of reducing the number of drawings and ease of description, respective portions included in the radiation detector 30 or a component thereof are shown in exemplary embodiments of FIG. 3 and subsequent figures. However, it should be understood that the radiation detector 30 may include a structure included in any of the embodiments described below, and does not necessarily include all of the structures in FIG. 3.

The radiation detector 30 according to some embodiments of the present disclosure includes a detector module 31. The detector module 31 is used to detect rays, for example, to detect X-rays emitted from a radiation source. The radiation detector 30 may further include a support frame 32. The support frame 32 is used to mount the detector module 31. The support frame 32 can perform heat conduction with the detector module 31. The radiation detector 30 may further include a temperature regulation module 33, as indicated by the dashed box in FIG. 3. The temperature regulation module 33 performs heat conduction with the support frame 32. In addition, the temperature regulation module 33 can further perform heat conduction with a mounting plate 34 on which the detector module 31 is mounted.

In some embodiments, the support frame 32 may include a heat-conducting material, so as to perform heat conduction with the temperature regulation module 33 and the detector module 31. In some embodiments, the heat-conducting material of the support frame 32 may include a metal or a metal alloy, such as aluminum or an aluminum alloy.

In some embodiments, the temperature regulation module 33 may include a temperature regulation element that uses electricity for cooling and heating. For example, as an example, the temperature regulation element that uses electricity for cooling and heating may include a thermoelectric cooler (TEC). The working principle of a TEC is based on the Peltier effect. When a direct current passes through a galvanic couple composed of two different semiconductor materials, a phenomenon of heat absorption at one end and heat release at the other end occurs at both ends of the galvanic couple. The smallest unit of a TEC is generally composed of a pair of N-type and P-type semiconductors, plus connecting electrodes, forming a cold end and a hot end. Under the action of an applied electric field, the current can carry heat from one end of the TEC to the other, thereby generating a “hot” side and a “cold” side of the TEC. When the current direction is reversed, the hot and cold ends of the TEC are switched, which can implement switching between heating and cooling functions.

In some embodiments, for example, when the radiation detector 30 is used in a CT imaging system, the mounting plate 34 may be a CT gantry back plate, such as a mounting plate for mounting the detector element 202 and/or the DAS 214 to the CT gantry 102 in FIG. 2. A metal or metal alloy material may be used for the mounting plate 34, and the mounting plate 34 not only has high heat conductivity and a large area, but also is connected to the gantry, further expanding the area for heat dissipation.

In a technical solution of the present disclosure, the support frame 32 is configured for mounting the detector module 31 and performing heat conduction separately with the temperature regulation module 33 and the detector module 31, to control the temperature of the detector module 31 via the temperature regulation module 33. Such a structural design simplifies the entire radiation detector 30. In addition, in a technical solution of the present disclosure, the mounting plate 34 originally used to mount the detector module 31 is used as a heat dissipation structure, which not only makes a heat dissipation area large and is beneficial for rapid diffusion of heat, but also eliminates the need for a separate heat sink, e.g., eliminates the need for structures such as additional fans or heat dissipation fins. In this way, the structure of the radiation detector 30 is further simplified, and the effect on the temperature regulation module 33 (for example, on the TEC) due to certain problems that may be caused by using a fan (for example, fan dust accumulation, vibration, and moisture condensation) is reduced. Therefore, the design of the present disclosure also improves the working reliability of the temperature regulation module 33 (for example, the TEC).

FIG. 4 is a schematic diagram of a temperature regulation module 33 according to some embodiments of the present disclosure. The temperature regulation module 33 may include a temperature regulation element (for example, a TEC) 331 that uses electricity for cooling and heating. For ease of description, the following will be mainly described using a TEC as the temperature regulation element that uses electricity for cooling and heating. The temperature regulation module 33 may further include a heat-conducting structure 332 on which the temperature regulation element (for example, the TEC) 331 is mounted. The heat-conducting structure 332 can not only be used to conduct heat, but can also be used to support the TEC 331. In some embodiments, the heat-conducting structure 332 may include a metal or a metal alloy, including but not limited to aluminum or an aluminum alloy, or the like.

Still referring back to FIG. 3, the temperature regulation module 33 may be disposed between the detector module 31 and the mounting plate 34. The temperature regulation module 33 is connected to the support frame 32 and conducts heat. The heat-conducting structure 332 of the temperature regulation module 33 can be connected to and perform heat conduction with the mounting plate 34.

In some embodiments of the present disclosure, the heat-conducting structure 332 can be fixedly connected to the support frame 32, so that a surface (the right-side surface shown in FIG. 3, the upper surface shown in FIG. 4) of the temperature regulation element (for example, the TEC) 331 is connected to the support frame 32 and conducts heat. For example, as shown in FIG. 3, by way of example and not limitation, the heat-conducting structure 332 can be fixedly connected to the support frame 32 by means of a fastener 335, so that the surface (the right-side surface shown in FIG. 3, the upper surface shown in FIG. 4) of the temperature regulation element (for example, the TEC) 331 is connected to the support frame 32 and conducts heat. The fastener 335 may include one or more fasteners 335. The fastener 335 may include a screw or other suitable fasteners.

As shown in FIG. 3 and FIG. 4, the TEC 331 is provided on a portion of a surface of the heat-conducting structure 332 facing the support frame 32. In some embodiments, the TEC 331 may be welded to the portion of the surface of the heat-conducting structure 332 facing the support frame 32. For example, FIG. 4 shows a welding part 334 for welding the TEC 331 to the heat-conducting structure 332. The use of welding can reduce heat resistance and improve heat transfer efficiency.

The heat-conducting structure 332 is thermally isolated from the support frame 32. In some embodiments of the present disclosure, the temperature regulation module 33 may further include a heat-insulating material 333. The heat-insulating material 333 may be provided on at least a part of a remaining portion of the surface of the heat-conducting structure 332 on which the TEC 331 is disposed, so that the heat-conducting structure 332 is thermally isolated from the support frame 32, to reduce or prevent an external environment from affecting the temperature of the detector module 31 by means of the mounting plate 34 and the heat-conducting structure 332, thereby reducing system energy consumption and improving temperature control accuracy. In some embodiments, the heat-insulating material 333 may include, but is not limited to, nylon or the like. Providing the heat-insulating material (for example, nylon) around at least a portion of the perimeter of the TEC 331 can also support the TEC 331 to prevent, or at least reduce, the risk of the TEC 331 being damaged by stress during mounting.

In a technical solution of the present disclosure, one side (the right side shown in FIG. 3) of the TEC 331 is used as a temperature control execution mechanism to control the temperature of the detector module 31, the other side (the left side shown in FIG. 3) of the TEC 331 is connected to the mounting plate 34 as a heat dissipation surface, and the heat-conducting structure 332 performs heat conduction with the mounting plate 34 but is thermally isolated from the support frame 32, so as to isolate heat transfer between the cold end and the hot end of the TEC 331, thereby more accurately controlling the temperature of the detector module 31. Moreover, because heat is dissipated by means of the mounting plate 34 (without using a fan or a heat dissipation fin) and heat transfer between the cold end and the hot end of the TEC 331 is isolated, the problem of high energy consumption caused by diffusion of a large amount of heat into the air by means of, e.g., a heat dissipation fin when the detector module 31 is heated can be avoided. Therefore, the technical solution of the present disclosure achieves more accurate temperature control of the detector module 31, and can reduce system energy consumption.

The temperature regulation module 33 is connected to and performs heat conduction with the support frame 32 at a first side (for example, the right side shown in FIG. 3). The temperature regulation module 33 is connected to and performs heat conduction with the mounting plate 34 at a second side (for example, the left side shown in FIG. 3). In some embodiments, the temperature regulation module 33 is directly connected to the mounting plate 34 at the second side (for example, the left side shown in FIG. 3). For example, at the second side (for example, the left side shown in FIG. 3) of the temperature regulation module 33, the heat-conducting structure 332 can be directly connected to the mounting plate 34 by means of a fastener (not shown), so that a surface of the left side shown in FIG. 3 of the heat-conducting structure 332 is connected to and performs heat conduction with the mounting plate 34. The fastener may include one or more fasteners. The fastener may include a screw or other suitable fasteners.

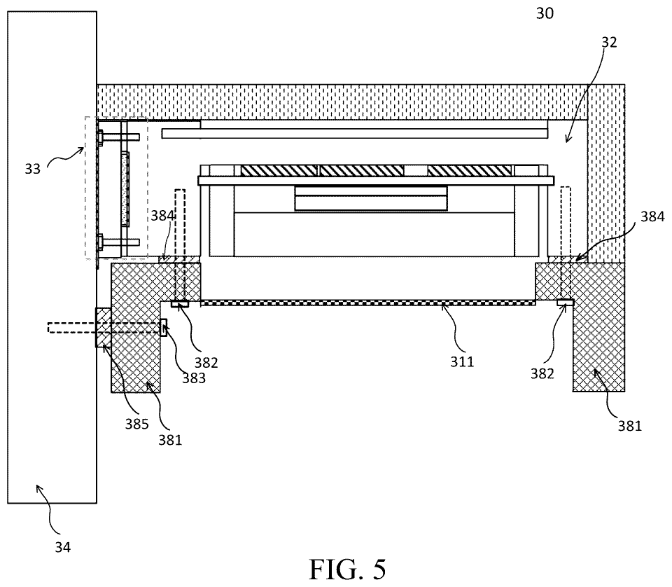

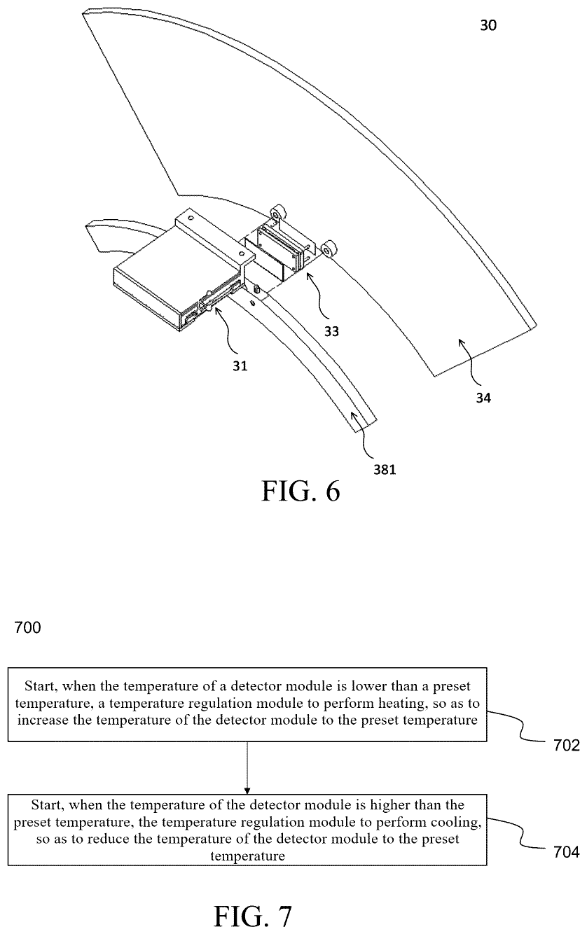

FIG. 5 and FIG. 6 show a schematic cross-sectional diagram and a three-dimensional schematic diagram, respectively, of a radiation detector 30 in a mounted state according to some other embodiments of the present disclosure. In some other embodiments, as shown in FIG. 5 and FIG. 6, the support frame 32 can indirectly connect the temperature regulation module 33 to the mounting plate 34 by means of a guide rail 381. In this case, a surface (for example, the left side surface of the heat-conducting structure 332) of the second side (for example, the left side shown in FIG. 3) of the temperature regulation module 33 is configured to be in contact (for example, in direct contact, or in indirect contact by means of a heat-conducting pad 35 as described below) with the mounting plate 34. As shown in FIG. 5, the support frame 32 and the guide rail 381 can be fixedly connected by means of a first fastener 382. One or more first fasteners 382 may be included. The first fastener 382 may include, but is not limited to, a screw. The guide rail 381 and the mounting plate 34 can be fixedly connected by means of a second fastener 383. One or more second fasteners 383 may be included. The second fastener 382 may include, but is not limited to, a screw.

One or more heat-insulating materials 384, such as a heat-insulating gasket, may be included between the support frame 32 and the guide rail 381. In addition, one or more heat-insulating materials 385, such as a heat-insulating gasket, may be included between the guide rail 381 and the mounting plate 34. The heat-insulating material can reduce the influence of the external environment, e.g., by means of the mounting plate 34, on the temperature of the detector module 31, and can improve the accuracy of control of the temperature of the detector module 31. In some embodiments, the thickness of the heat-insulating material 385 between the guide rail 381 and the mounting plate 34 is set to implement or ensure that the surface of the second side (for example, the left side shown in FIG. 5) of the temperature regulation module 33 is in contact (in direct contact or in indirect contact by means of the heat-conducting pad 35) with the mounting plate 34, so that the heat-conducting structure 332 can perform heat conduction with the mounting plate 34.

In some embodiments of the present disclosure, the radiation detector 30 may further include a first heat-conducting pad 35 disposed between the mounting plate 34 and the temperature regulation module 33. The first heat-conducting pad 35 may have elasticity, and may be compressed during mounting of the TEC 331. For example, in some embodiments, a groove may be provided on a side of the mounting plate 34 facing the heat-conducting structure 332, and the heat-conducting pad 35 may be placed inside the groove. In some embodiments of the present disclosure, the radiation detector 30 may further include a second heat-conducting pad 36 disposed between the support frame 32 and the temperature regulation module 33. The second heat-conducting pad 36 may have elasticity and may be compressed during mounting of the TEC 331. For example, in some embodiments, a groove may be provided on a side of the support frame 32 facing the TEC 331, and the heat-conducting pad 36 may be placed inside the groove. In some exemplary embodiments, the depth of the groove may be set to 0.5 mm to 2 mm. Elastic characteristics of the heat-conducting pad 35 and/or the heat-conducting pad 36 can be used to protect the TEC 331 from being damaged during mounting.

In embodiments including the heat-conducting pad 35 and/or the heat-conducting pad 36, the thickness of the heat-insulating material 385 between the guide rail 381 and the mounting plate 34 is further set such that the heat-conducting pad 35 and/or the heat-conducting pad 36 has a reasonable compression ratio, so as to implement or ensure that the surface of the second side (for example, the left side shown in FIG. 5) of the temperature regulation module 33 is in contact (for example, in indirect contact by means of the heat-conducting pad 35) with the mounting plate 34.

In some embodiments, at least a portion of the outer perimeter of at least one of the temperature regulation module 33 and the support frame 32 is covered with a heat-insulating material 40, so as to reduce the influence of the external environment on the detector module 31, which can improve the accuracy of control of the temperature of the detector module 31. In some embodiments, the heat-insulating material 40 may include heat-isolating foam.

Heat generated by the TEC 331 (heat generated by heating or cooling) can be conducted to the detector module 31 by means of the support frame 32, or by means of the support frame 32 and a heat-conducting pipe 37, thereby controlling the temperature of the detector module 31. As shown in FIG. 3, the heat-conducting pipe 37 may be an elongated pipe, e.g., a metal pipe such as a copper pipe. The heat-conducting pipe 37 may be connected or mounted to the support frame 32. The heat-conducting pipe 37 may include or contain a liquid (for example, water or another suitable liquid). One end (for example, the left end shown in FIG. 3) of the heat-conducting pipe 37 may be close to the TEC 331. Using a heating mode as an example, when the TEC 331 generates heat, the heat can be conducted to an end of the heat-conducting pipe 37 close to the TEC 331 (for example, the left end shown in FIG. 3). The liquid in that end of the heat-conducting pipe 37 absorbs heat and evaporates, diffuses, and reaches the other end (for example, the right end shown in FIG. 3) of the heat-conducting pipe 37. Because the temperature at the other end is low, the liquid condenses and returns to a liquid state, and becomes a part of the liquid in the heat-conducting pipe 37 again. In this process, heat is transferred from an end of the heat-conducting pipe 37 close to the TEC 331 to the other end of the heat-conducting pipe 37 distant from the TEC 331, thereby implementing heat transfer from the TEC 331 to the detector module 31. Because heat transferred by means of evaporation is much greater than heat transferred by means of conduction and convection, evaporation of the liquid in the heat-conducting pipe 37 causes heat to be transferred quickly from one end to the other end, which can increase a heat transfer speed.

In some embodiments of the present disclosure, the radiation detector 30 may further include a temperature sensor 39. The temperature sensor 39 may be configured to measure the temperature of the detector module 31. In some embodiments of the present disclosure, the temperature sensor 39 may be disposed close to a heat emitting element of the detector module 31. The temperature sensor 39 may be disposed on or in a circuit board 315 of the detector module 31 as described below, so as to measure the temperature of the circuit board 315 and a detector element 313, a signal processing circuit 314, and the like that are attached to the circuit board 315.

The temperature regulation module 33 may be configured to perform heating when the temperature of the detector module 31 is lower than a preset temperature, so as to increase the temperature of the detector module 31 to the preset temperature. The temperature regulation module 33 may further be configured to perform cooling when the temperature of the detector module 31 is higher than the preset temperature, to reduce the temperature of the detector module 31 to the preset temperature.

In some practical uses, it is generally chosen to set the TEC to be in a cooling operation mode. In some embodiments of the present disclosure, the preset temperature of the detector module 31 is set to be greater than or equal to a maximum temperature allowed inside an imaging system employing the radiation detector 30. In this way, in a general case, the TEC 331 is mainly in a heating operation mode. In the heating operation mode, a coefficient of performance (COP) is always greater than 1, energy utilization efficiency is high, and energy consumption of the system is reduced. In addition, in the heating operation mode, a cooling end of the TEC 331 is connected to the mounting plate 34 (for example, the back plate of the gantry of the imaging system), and the large heat dissipation area of the mounting plate 34 can eliminate, or at least reduce, the risk of condensation. In other cases, for example, when the imaging system is operating under a heavy load in extreme working conditions of high altitudes, high room temperatures and/or high power consumption, the temperature of the detector module 31 is high, and in this case, the TEC 331 may be controlled to work in a cooling state. Because a preset working temperature of the detector module 31 is greater than or equal to the maximum temperature allowed inside the imaging system employing the radiation detector 30, and accordingly higher than the air temperature inside the mounting plate 34 or the connected, for example, gantry of the imaging system, there is no risk of moisture condensation.

In some embodiments, for example, when the imaging system is a CT imaging system, a maximum temperature allowed inside the CT imaging system may be a temperature value in the range of, for example, 35° C. to 40° C. The working temperature of the detector module 31 may be set to be greater than or equal to the temperature value. For example, as an example, when the maximum temperature allowed inside the CT imaging system is 36° C., the working temperature of the detector module 31 may be set to be greater than or equal to 36° C. In general, the CT imaging system is in an environment having a temperature of greater than 20 degrees. Therefore, the working temperature of the detector module 31 may be set to be greater than or equal to 36° C., so that the TEC 11 is mainly in the heating operation mode, which can achieve the beneficial effects in the above aspects.

In some cases, the TEC 331 may need to switch between a heating mode and a cooling mode. In some embodiments of the present disclosure, the temperature regulation module 33 is configured to allow a predetermined switching time to pass when switching between heating and cooling, so as to protect the TEC 331. In some embodiments of the present disclosure, the predetermined switching time may include several seconds. For example, the predetermined switching time may include a value in the range of 4 seconds to 10 seconds (including endpoint values). For example, the predetermined switching time may include a value in the range of 5 seconds to 7 seconds (including endpoint values).

Referring again to FIG. 3, in some embodiments of the present disclosure, the detector module 31 may include one or a plurality of: a window 311, used to allow X-rays to pass therethrough; a collimator 312, used to collimate or homogenize X-rays, and prevent or reduce ray scattering; a detector element 313, wherein, for example, when radiation rays are X-rays, the detector element 313 may include an element 313a (such as a scintillator) that first converts the X-rays into visible light, and an element 313b (such as a photodiode) that further converts the visible light into an electrical signal, or alternatively, the detector element 313 may include a photon counting detector element or other types of element that directly converts the X-rays into electrical signals; and a signal processing element 314, wherein the signal processing element 314 may include an analog-to-digital conversion circuit (such as an AD chip) for converting an analog signal generated by the detector element 313 into a digital signal for subsequent processing (for example, image reconstruction).

In some embodiments, as shown in FIG. 3, the detector module 31 may include a flat plate-shaped detector circuit board 315. In some embodiments, the flat plate-shaped detector circuit board 315 may be connected to the support frame 32. For example, as an example, both sides of the flat plate-shaped detector circuit board 315 can be inserted or embedded into the support frame 32, or fixed to the inside or surface of the support frame 32 by other means, so as to achieve a fixed connection with the support frame 32. The detector module 31 may include a detector element 313 mounted on a surface of a side of the detector circuit board 315 facing a ray source generating X-rays (for example, a lower surface of the detector circuit board 315 shown in FIG. 3). The detector module 31 may further include a signal processing element 314 mounted on a surface of a side of the detector circuit board 315 facing away from the ray source (for example, an upper surface of the detector circuit board 315 shown in FIG. 3). As shown in FIG. 3, in some embodiments, the support frame 32 may include a flat plate-shaped main body portion covering the detector module 31 and performing heat conduction with the detector module 31.

In some embodiments, heat generated by the detector module 31 may mainly be heat generated by the signal processing element 314 (for example, a signal processing ASIC) during operation. Therefore, the support frame 32 can perform heat conduction with the signal processing element 314. For example, the flat plate-shaped detector circuit board 315 may be configured such that the signal processing element 314 mounted on the surface of the flat plate-shaped detector circuit board 315 is connected (including directly connected or indirectly connected) to an inner surface of the support frame 32. In some embodiments, the inner surface of the support frame 32 may be in direct contact and perform heat conduction with the signal processing element 314.

In the embodiments of the present disclosure, because the detector element 313, the signal processing element 314, and the collimator 312, for example, included in the detector module 31 are maintained at the same temperature, a change in an effective imaging area caused by a temperature difference between the detector element 313, the signal processing element 314, and the collimator 312 can be reduced, and performance of the imaging system can be further improved.

The present disclosure further provides an imaging system. The imaging system includes a radiation source configured to emit radiation rays. The imaging system may include a CT imaging system 100, as described with reference to FIG. 1 and FIG. 2. Accordingly, the radiation source may include an X-ray radiation source 104 as described in FIG. 1 and FIG. 2.

The imaging system may further include a radiation detector according to any embodiment of the present disclosure, such as any radiation detector 30 in the present disclosure. The imaging system may include one or more radiation detectors. The imaging system may further include a gantry. The gantry may include a gantry 102 as described with reference to FIG. 1 and FIG. 2.

The radiation detector (such as any radiation detector 30 in the present disclosure) may be disposed inside the gantry (for example, the gantry 102) by means of a mounting plate 34.

FIG. 7 shows a temperature control method 700 for a radiation detector according to some embodiments of the present disclosure. The temperature control method 700 may include: starting, when the temperature of a detector module 31 is lower than a preset temperature, a temperature regulation module 33 to perform heating, so as to increase the temperature of the detector module 31 to the preset temperature. The temperature control method 700 may further include: starting, when the temperature of the detector module 31 is higher than the preset temperature, the temperature regulation module 33 to perform cooling, so as to reduce the temperature of the detector module 31 to the preset temperature.

The temperature control method 700 may further include: setting the preset temperature of the detector module 31 to be greater than or equal to a maximum temperature allowed inside an imaging system employing the radiation detector. In some embodiments, for example, when the imaging system is a CT imaging system, a maximum temperature allowed inside the CT imaging system may be, e.g., a temperature value in the range of 35° C. to 40° C., and the working temperature of the detector module 31 may be set to be greater than the temperature value. For example, as an example, when the maximum temperature allowed inside the CT imaging system is 36° C., the working temperature of the detector module 31 may be set to be greater than or equal to 36° C.

The temperature control method 700 may further include: configuring the temperature regulation module 33 to allow a predetermined switching time to pass when switching between a heating mode and a cooling mode. The predetermined switching time can be set to protect the TEC 331 from being damaged. In some embodiments, the predetermined switching time may include several seconds. For example, the predetermined switching time may include a value in the range of 4 seconds to 10 seconds (including endpoint values). For example, the predetermined switching time may include a value in the range of 5 seconds to 7 seconds (including endpoint values).

The steps of the temperature control method 700 described above with reference to FIG. 7 are not intended to limit the order of execution of the method 700. One or a plurality of steps of the method 700 may be performed in a different order according to actual situations.

Therefore, a person skilled in the art can make appropriate modifications and adjustments to the embodiments described in detail above without departing from the spirit and gist of the present invention. Therefore, it is intended that the claimed subject matter is not limited to only particular examples disclosed, and the claimed subject matter may also include all implementations that fall within the scope of the appended claims and equivalents thereof.

1. A radiation detector, comprising:
a detector module, the detector module being used to detect X-rays;
a support frame, used to mount the detector module, the support frame performing heat conduction with the detector module; and
a temperature regulation module, the temperature regulation module performing heat conduction with the support frame, the temperature regulation module being used to perform heat conduction with a mounting plate on which the detector module is mounted, and the temperature regulation module comprising a temperature regulation element that uses electricity for cooling and heating.

2. The radiation detector according to claim 1, wherein the temperature regulation element comprises a thermoelectric cooler (TEC).

3. The radiation detector according to claim 1, wherein the temperature regulation module is disposed between the detector module and the mounting plate, the temperature regulation module is connected to the support frame and conducts heat, the temperature regulation module comprises a metal heat-conducting structure on which the temperature regulation element is mounted, the metal heat-conducting structure performs heat conduction with the mounting plate, and the metal heat-conducting structure is thermally isolated from the support frame.

4. The radiation detector according to claim 3, wherein the metal heat-conducting structure is fixedly connected to the support frame, the temperature regulation element is disposed on a portion of a surface of the metal heat-conducting structure facing the support frame, and a heat-insulating material is provided on at least part of a remaining portion of the surface of the metal heat-conducting structure, to thermally isolate the metal heat-conducting structure from the support frame.

5. The radiation detector according to claim 4, wherein the temperature regulation element is welded to the portion of the surface of the metal heat-conducting structure facing the support frame.

6. The radiation detector according to claim 1, further comprising a temperature sensor, the temperature sensor being configured to measure the temperature of the detector module, and
the temperature regulation module being configured to:
perform heating when the temperature of the detector module is lower than a preset temperature, so as to increase the temperature of the detector module to the preset temperature; and
perform cooling when the temperature of the detector module is higher than the preset temperature, so as to reduce the temperature of the detector module to the preset temperature.

7. The radiation detector according to claim 6, wherein the preset temperature is greater than or equal to a maximum temperature allowed inside an imaging system employing the radiation detector.

8. The radiation detector according to claim 6, wherein the temperature regulation module is configured to allow a predetermined switching time to pass when switching between heating and cooling.

9. The radiation detector according to claim 1, further comprising at least one of:
a first elastic heat-conducting pad disposed between the mounting plate and the temperature regulation module; and
a second elastic heat-conducting pad disposed between the support frame and the temperature regulation module.

10. The radiation detector according to claim 1, wherein
the temperature regulation module is fixedly connected to the support frame at a first side; and
the temperature regulation module is directly connected to the mounting plate at a second side.

11. The radiation detector according to claim 1, wherein
the temperature regulation module is fixedly connected to the support frame at a first side; and
the support frame is connected to the mounting plate by means of a guide rail, and a surface of a second side of the temperature regulation module is in contact with the mounting plate, wherein
the support frame is fixedly connected to the guide rail by means of a first fastener, and the guide rail is fixedly connected to the mounting plate by means of a second fastener.

12. The radiation detector according to claim 11, wherein a heat-insulating material is comprised between the support frame and the guide rail, and a heat-insulating material is comprised between the guide rail and the mounting plate, wherein
the thickness of the heat-insulating material between the guide rail and the mounting plate is configured such that a surface of the first side of the temperature regulation module is in contact with the mounting plate.

13. The radiation detector according to claim 1, wherein the detector module comprises a flat plate-shaped detector circuit board, a detector element mounted on a surface of a side of the detector circuit board facing a ray source generating rays, and a signal processing element mounted on a surface of a side of the detector circuit board facing away from the ray source; and
the detector circuit board is mounted on the support frame, and the support frame comprises a flat plate-shaped main body portion covering the detector module and performing heat conduction with the detector module.

14. The radiation detector according to claim 1, wherein at least a portion of the outer perimeter of at least one of the temperature regulation module and the support frame is covered with a heat-insulating material.

15. The radiation detector according to claim 1, further comprising a heat-conducting pipe connected to the support frame, one end of the heat-conducting pipe being adjacent to the temperature regulation element, and the heat-conducting pipe comprising a liquid and being configured to conduct heat generated by the temperature regulation element to the support frame.

16. An imaging system, comprising:
a radiation source, the radiation source being configured to emit X-rays;
a radiation detector, the radiation detector including:
a detector module, the detector module being used to detect X-rays;
a support frame, used to mount the detector module, the support frame performing heat conduction with the detector module; and
a temperature regulation module, the temperature regulation module performing heat conduction with the support frame, the temperature regulation module being used to perform heat conduction with a mounting plate on which the detector module is mounted, and the temperature regulation module comprising a temperature regulation element that uses electricity for cooling and heating; and
a gantry, wherein the radiation detector is disposed inside the gantry by means of the mounting plate.

17. A temperature control method for a radiation detector, the method comprising:
starting, when the temperature of a detector module is lower than a preset temperature, a temperature regulation module to perform heating, so as to increase the temperature of the detector module to the preset temperature; and
starting, when the temperature of the detector module is higher than the preset temperature, the temperature regulation module to perform cooling, so as to reduce the temperature of the detector module to the preset temperature.

18. The temperature control method according to claim 17, further comprising:
setting the preset temperature of the detector module to be greater than or equal to a maximum temperature allowed inside an imaging system employing the radiation detector.

19. The temperature control method according to claim 17, further comprising:
configuring the temperature regulation module to allow a predetermined switching time to pass when switching between heating and cooling.
